(19) United States
(12) Patent Application Publication
(10) Pub. No.: US 2018/0200470 A1
(43) Pub. Date: Jul. 19, 2018

(54) DEVICE AND SYSTEM FOR HEATING RESPIRATORY CONDUIT

(71) Applicant: Ventific Holdings Pty Ltd., New South Wales (AU)

(72) Inventors: Fergus Henry Dixon, New South Wales (AU); Michael David Hallet, New South Wales (AU); Roger Foote, New South Wales (AU)

(21) Appl. No.: 15/742,782

(22) PCT Filed: Jun. 30, 2016
(86) PCT No.: PCT/AU2016/050561

§ 371 Date: Jan. 8, 2018

(30) Foreign Application Priority Data
Jul. 8, 2015 (AU) 2015902688
Nov. 9, 2015 (AU) 2015904595

(51) Int. Cl. A61M 16/10; A61M 16/00; A61M 16/16

(52) U.S. Cl. CPC A61M 16/1095; A61M 16/0066; A61M 2205/3368; A61M 16/16; A61M 16/024

(57) ABSTRACT

The present invention provides a system for heating a respiratory conduit positioned between a PAP device and a patent interface, wherein the system comprises: a first thermistor encapsulated within the conduit proximal to a first end of the conduit which is proximal to the patient interfaces; a second thermistor encapsulated within the conduit proximal to a second end of the conduit which is proximal to the PAP device; a heater is encapsulated within the conduit; and wherein the first and second thermistor are electrically isolated from the heater within the conduit.

### TECHNICAL FIELD

The present invention relates to a system, method or device adapted to control the heating of respiratory conduit connected between a Positive Airway Pressure (PAP) machine and face mask. This invention is preferably adapted for use with respiratory conduit that includes a heating element to heat air supplied to a patient.

### BACKGROUND

Previously, there have been many attempts to provide for a system or device that adequately controls the heating of respiratory conduit to maintain humidity levels of the air in the conduit. Previously, respiratory conduit without heating elements mounted in the walls of the conduit has led to condensation of the water from humidifiers forming on the interior wall of the conduit.

This condensation event is commonly called ‘rain-out. Rain out may lead to poor humidification of the air received by the patient. Additionally, the excess condensation may pool and fall back into the PAP machine. Also, bacteria and fungus may contaminate or grow on the interior walls of the conduit leading to further health problems for the patient.

US Published Patent Application No. 2013/0333701—Herron describes a heating element integrated into the walls of a respiratory conduit. The conduit in this publication is for use with a Continuous Positive Airways Pressure (CPAP) device. The publication is focused on detailing a coupling system for electrically connecting the heating element via co-axial style connector to an electrical inducer with provides electrical current to the heating element. The control system of this publication is a manually operated switch which is problematic for patients to operate whilst asleep.

U.S. Pat. No. 6,953,354—Edirisuriya et al. describes a coupling system for connecting respiratory conduit between a patient interface and PAP device, wherein the conduit includes a series of specialised locking and securing connectors.

US Published Patent Application No. 20080105257—Klasek et al discloses a respiratory conduit with a heated element. The heating element is a specialised element that includes heating element in an elongated tape configuration.

U.S. Pat. No. 8,733,349—Bath et al discloses a respiratory conduit heating device and system for use with CPAPs and Variable Positive Airways Pressure (VPAP) machines. The device includes a heater assembly integrated into a respiratory conduit. The heating system includes three wires within the conduit. The system adapts and control the heating of the conduit by automatically monitoring the temperature in the conduit by the use of a single thermistor mounted proximal to the face of the patient or facemask being used by the patient. The thermistor feeds back changes in voltage drop to a controller which then interprets and translates the voltage drop into temperature data. The calculated temperature data is then used by the electronic controller to amend the heating pattern or instructions sent to a humidifier connected to the system.

There are a few disadvantages with this system including the over-reliance on a single thermistor that allows this previously disclosed system to be subject to baseline shift leading to inaccurate results over prolonged usage. Further the reliance on using voltage drop as an indicator of a temperature from the thermistor may also change over time leading to further baseline shifts in the comparison of temperature to voltage characteristics of the thermistor.

Any discussion of the prior art throughout the specification should in no way be considered as an admission that such prior art is widely known or forms part of common general knowledge in the field.

### SUMMARY

### Problems to be Solved

It is an object of the present invention to provide for a system, process or device that allows for automatic control of a heating element within a respiratory conduit.

Further, it is an object of the present invention to limit or ameliorate rain-out events whilst reducing the risks of overheating the air or conduit due to improper control of heating element. A further object of the present invention may be improved patient compliance with using the associated PAP device.

A further object may be to improve patient comfort when using the associated PAP system.

It is an object of the present invention to overcome or ameliorate at least one of the disadvantages of the prior art, or to provide a useful alternative.

### Means For Solving the Problem

A first aspect of the present invention may relates to: a control system for a heated respiratory conduit positioned between a PAP device and a patient interface, wherein the control system comprises: a power supply to provide power to at least one heating element in the conduit; an over temperature control circuit to prevent overheating of the heating element; a heating control circuit configured to control heating element within the respiratory conduit to obtain a predetermined temperature; and a sensing circuit including at least a first thermistor positioned in the respiratory conduit proximal to a patient interface and configured to indicate a second temperature, and a second thermistor positioned in the heated conduit proximal to a PAP device and configured to indicate a first temperature.

Preferably, the heating control circuit includes: at least a first rail, second rail, third rail and fourth rail.

Preferably, the first thermistor may be electrically connected to the third rail.

Preferably, the second thermistor is electrically connected to the second rail.

Preferably, a current may be applied to the second and third rail by a controller, when in use.

Preferably, the system compares the second temperature to the first temperature added to a predefined offset temperature, and wherein second temperature is lower than the first temperature added to the predefined offset temperature activates the heating control circuit.

Preferably, the predefined offset temperature is between 0 and 5 degrees Celsius.

Preferably, the system measures current usage across the rails to determine temperature at the first and second thermistor.

Preferably, the system uses a current source to power at the first and second thermistor to determine temperature proximal to the respective the first or second thermistor.

Preferably, the first rail may be connected to P channel transistor switch.

Preferably, the fourth rail may be connected to N channel transistor switch.

A second aspect of the present invention may include a process for controlling the temperature of air inside a respiratory conduit positioned between a PAP device and a patient interface, wherein the process comprises the following steps: a first temperature is measured from a second thermistor positioned distal to the patient interface and a second temperature is detected from a first temperature sensor positioned proximal to the PAP device; and comparing the second temperature to the first temperature added to an predefined offset temperature, and wherein second temperature is calculated to be lower than the first temperature added to an predefined offset temperature, at least one heating element within the conduit is activated.

Preferably, the predefined offset temperature is between 0 and 5 degrees Celsius.

Preferably, the process may include an additional step, wherein once the heating element is activated, heat is continuously applied to the conduit until the measured value of the second temperature exceeds the initial temperature of second temperature added to the temperature swing value.

Preferably, the preferred process measures the current used by the heating element and first thermistor and the second thermistor.

Preferably, the process measures the current used by the heating element and the voltage across the first thermistor and the second thermistor generated by the two current sources.

In another aspect of the present invention, there may be provided a system for heating a respiratory conduit positioned between a PAP device and a patent interface, wherein the system comprises: a first thermistor encapsulated within the conduit proximal to a first end of the conduit which is proximal to the patient interface; a second thermistor encapsulated within the conduit proximal to a second end of the conduit which is proximal to the PAP device; a heater is encapsulated within the conduit; and wherein the first and second thermistor are electrically isolated from the heater within the conduit.

Preferably, the system may include a humidifier mounted within or on the patient interface.

Preferably, the preferred conduit includes at five electrical conductive pins for connecting the components encapsulated within the conduit; and wherein the components include: the heater, the first thermistor and the second thermistor.

Preferably, the first thermistor may measure temperature proximal to the patient interface.

Preferably, the second thermistor may measure temperature proximal to the PAP device or blower.

Preferably, the first and second thermistors are electrically connected to a first circuit; and the heater is electrically connected to a second circuit and wherein the first and second circuits are not electrically connected within the conduit.

Preferably, the humidifier may be in the form of a HME (humidity moisture exchanger) module mounted within the patient interface.

Preferably the conduit comprises at least four wires encapsulated within an outer surface of the conduit in a helix.

Preferably, the conduit includes at least two spaced apart wiring sets encapsulated within an outer surface of the conduit in a helix.

In the context of the present invention, the words ‘comprise, ‘comprising and the like are to be construed in their inclusive, as opposed to their exclusive, sense, that is in the sense of ‘including, but not limited to.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 depicts a schematic block diagram of PAP system according to a preferred embodiment;

FIG. 2 depicts a second schematic block diagram of PAP system according to a further preferred embodiment;

FIG. 3 depicts a third schematic block diagram of PAP system according to a further preferred embodiment;

FIG. 4 depicts a front perspective view of an embodied respiratory conduit for use with or as part of a preferred embodiment of the present invention;

FIG. 5 depicts a block diagram of a portion of a system forming part of a preferred embodiment;

FIG. 6 is a flowchart depicting a process or system used or employed by a preferred embodiment;

FIG. 7 depicts a block diagram of a portion of a system forming part of a second preferred embodiment; and

FIG. 8 is a perspective view of a section respiratory conduit forming part of a preferred embodiment of the present invention;

FIG. 9 is an enlarged sectional view of part of the respiratory conduit shown in FIG. 8; and

FIG. 10 is a further embodiment depicting a respiratory conduit.

### DESCRIPTION OF THE INVENTION

Preferred embodiments of the invention will now be described with reference to the accompanying drawings and non-limiting examples.

FIG. 1 depicts a first preferred embodiment of the present invention wherein a Positive Airways Pressure (PAP) device 1 has been connected to a patent interface 2 using a respiratory conduit 3. The PAP device 1 is preferably adapted to apply air at a pressure of greater than 1 ATM to the airways of a patient. The PAP device 1 preferably includes: a controller which controls and varies the pumping speed; an air pump and motor driven by the controller; and a power source.

The power source (not shown in FIG. 1) may be connected to mains supply or low voltage AC adapter or battery supply depending on the application. The power source provides power to the controller and motor which is turn attached to the pump. The controller varies the speed of rotation of the motor to vary the pump output. Preferably, the pump is a centrifugal pump capable of delivering pressure airway to the patient via the respiratory conduit 3 then to the patient interface 2 which in turn is adapted to seal against the face of the patient to delivered pressurised air to the airways of the patient to prevent or partially inhibit snoring or sleep apnea.

The respiratory conduit 3 is preferably configured to be a flexible pipe or tubing to carry pressurised air. These types of conduit are usually constructed of flexible plastic or polymer and may include ribbing or strength reinforcing features in its surface to prevent crimping, occlusion, or accidental damage.

The patient interface 2 may be a relatively rigid mask adapted to be worn on the face of the patient. Typically, patient interface 2 may be adapted to the shape and configuration to comfortably accommodate the faces of different users and anatomic facial shapes. The mask may include: full face masks covering nose and mouth of patient; nasal masks which are adapted to cover the nose of the patient; or nasal tubing or pillows configurations adapted to engage and seal against the openings of the nose.

Preferably, the pump may be referred to as a flow generator and may be configured to generate a flow of breathable gas (preferably air and/or oxygen depending on the particular treatment type) having a pressure of about 2-30 cm H₂O.

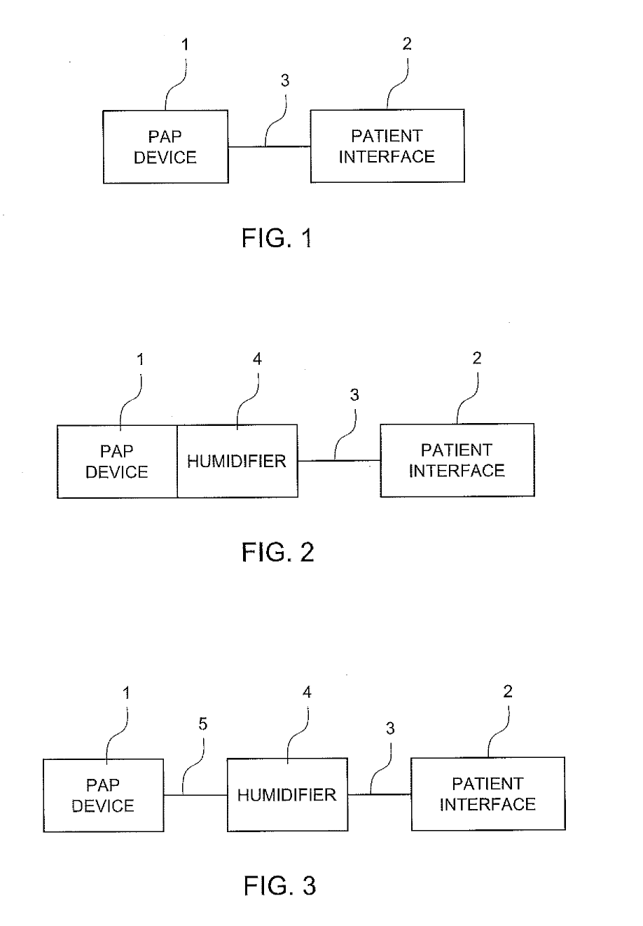

FIG. 2 depicts an improved design and configuration over the embodiment shown in FIG. 1, whereby the PAP device 1 includes a humidifier 4. The humidifier includes a water reservoir with a heating pad or plate. The heating pad is usually mounted or positioned directly in contact with the reservoir and usually below it. Wherein an electrical current is applied to the heating pad, the heating pad heats the water in the reservoir and partially evaporates some of the water into the air path exiting the pump.

The humidifier functions to increase the humidity in the air path of air exiting the pump and being pumped into the respiratory conduit 3. Generally, the heated humidifier is used to provide sufficient humidity and temperature to the air so that the patient will be comfortable. In such an embodiment, the respiratory conduit may be heated to heat the gas and prevent ‘rain-out or condensation forming on the inside of the conduit as the gas is supplied to the patient. In this arrangement, the respiratory conduct may include one or more wires or sensors associated with this heating.

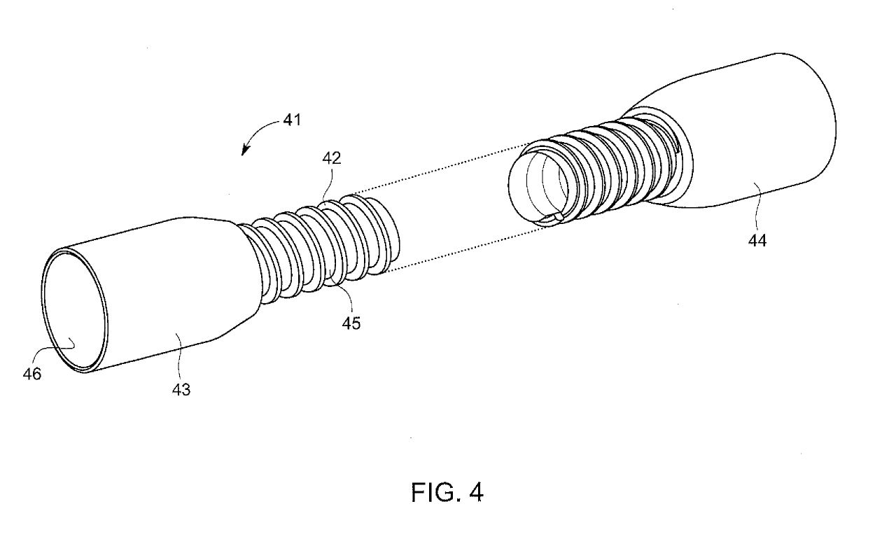

FIG. 3 is similar to FIGS. 1 and 2, however in this configuration, the PAP device 1 and humidifier 4 are not integrated together and have been separated. The humidifier 4 is still located within the air path downstream of the flow generator, but the humidifier may be an accessory module that can be removed or added to the circuit shown in FIG. 3 in accordance with the desires and needs of the either the patient or the clinician of the patient.

In FIG. 3, a second respiratory conduit 5 has been added to join and connect between the PAP device 1 and the humidifier 4. The first respiratory conduit 3 is retained to link between the humidifier 4 and the patient interface 2 as per the description associated with FIG. 2.

FIG. 4 depicts a front perspective view of a respiratory conduit 41 capable being used with or as part of a preferred embodiment of the present invention. Preferably, the conduit 41 is in the form of a flexible tube 45 having a first and second ends. The first end includes a first cuff portion 44 adapted for engagement to a patent interface 2.

The second end preferably includes a second cuff portion 43 adapted to engage the outlet of either PAP device 1 or the humidifier 4 depending on the preferred configuration.

The first and second cuff portions 44 and 43 are preferably polymeric and elastic in nature. The cuff portions may be selectively pushed over a mating portion or spigot of the respective item that it to be attached to. The elastic nature of the cuff portions may allow the cuff to expand in diameter to receive the aforementioned mating portion (which is usually a protrusion of the respective connecting item). The elastic nature of the cuff portion also al lows the cuff to retract against the outer walls of the respective mating portion to secure the conduit and the seal an air path. The cuff portions may be constructed of rubber, latex or silicone polymer.

Preferably, the respiratory conduit 41 is adapted to include a bore 46 running longitudinally through the central axis of the conduit. The bore 46 is adapted to carry pressurised air from the flow generator or humidifier to the patent interface. The air path formed within the conduit is sealed within the conduit and at the first and second ends 44, 46 when in use.

The conduit 41 is preferably flexible and may be bent to conform to the movements or positioning of the patient to which the patient interface is attached. Preferably, the conduit 41 includes a reinforcement means 42 attached to the tubing 45 between the first and second end. The reinforcement means 42 is preferred to being in the form of a helical rib rotated around the circumference of the tubing 45. The rib provides additional support to the walls of the tubing 45.

Preferably, the rib may also include a wiring set wherein the wiring set forms a heating circuit encapsulated within the walls of the conduit 41. Preferably, the heating circuit may comprise at least 2 wires running within the rib. When an electrical current is applied to the said wires, resistance in the wiring causes heat. The heat is applied to the tubing 45 which in turn heats the air within the air path in the bore 46. The heating is preferably adapted to prevent ‘rain-out within the respiratory conduct 41 when in use.

Preferably, the second cuff portion 43 may be adapted to include electrical contacts which mate with corresponding electrical contacts on the PAP device or humidifier. These electrical contacts may allow the controller of the PAP device to vary the current applied to the wire set to reduce or increase heating in accordance with safe operating procedures.

Preferably, the respiratory conductor may be conducted of flexible polymeric materials such as nylon or other plastics.

Preferably, the respiratory conduit may include a first and second thermistor (not shown in FIG. 4), which can fed temperature information to the PAP device controller. Preferably, the thermistor may be encapsulated within the first and/or second cuff portions. Alternate embodiments may allow for the thermistors to be positioned and encapsulated within the walls of the flexible conduct between the two cuff sections. However, the preferred positioning would be to include at least one thermistor proximal to the PAP device and at least a second thermistor proximal to the patient interface. Also preferably, the thermistors may be mounted in molded extensions entering the airflow path of the conduit from the inner walls of the conduit. More preferably, the thermistor may be positioned on an extension generally proximal to the central axis of the conduit. The extension may be in the form or shape of a tooth like member or crosshair member or beam like member.

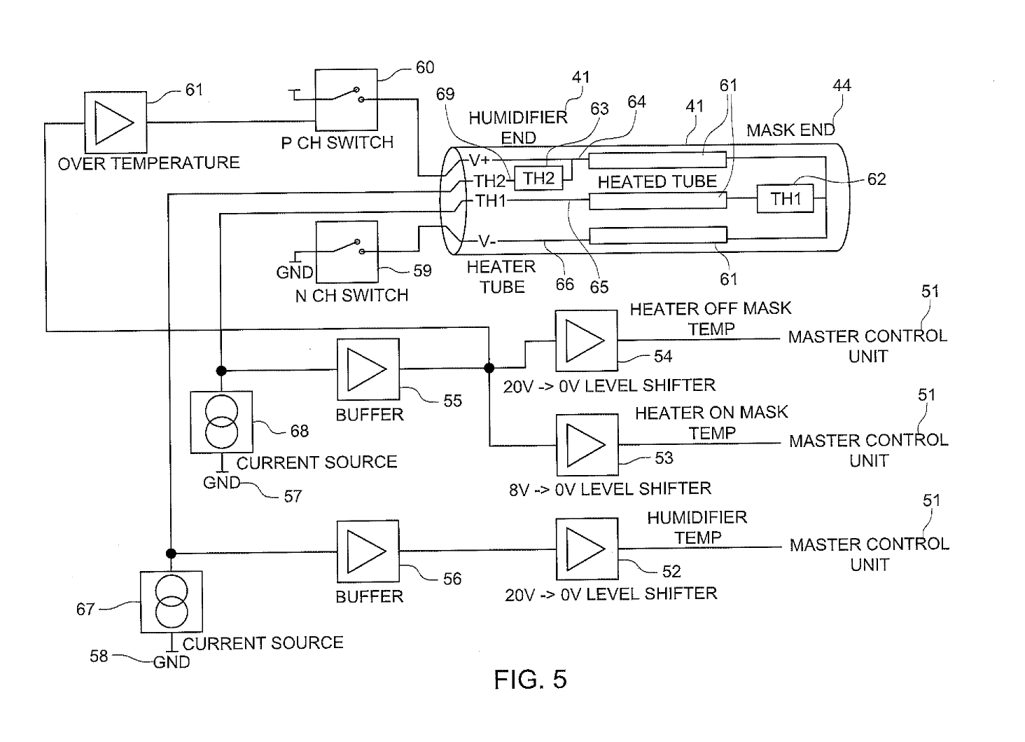

FIG. 5 is a block diagram of the electrical components of the heating system, process or device forming at least part of a preferred embodiment of the present invention. In this figure, the respiratory conduit 41 has been shown to include a four wire configuration. Each one of the wires forms either a first rail 64, second rail 69, third rail 65, or fourth rail 66.

The first, third, and fourth rails all include an additional heating element 61 which actively heats the conduit 41 when current is applied to these rails.

The system of FIG. 5 is primarily used to feedback information to the controller of the PAP device 1. In FIG. 5, the controller is represented as Master Control Unit 51. The Master Control Unit 51 monitors the temperature of the air path in the conduit and activates the heating circuit based on the temperature information. The Master Control Unit 51 may be integrated into the humidifier or the PAP device. FIG. 5 depicts a sensing circuit forming part of an overall preferred embodiment of the present invention.

A first thermistor TH1 or 62 is positioned in series on the third rail 65. The first thermistor 62 is preferably encapsulated near to or proximal to the end of the conduit connecting to the patient interface. The second thermistor TH2 or 63 is preferably encapsulated and positioned proximal to or near to the PAP device or humidifier.

Preferably, an electrical current may be applied by power sources 67 and 68. The alternate side of the power supply is connected the respective ground connections 58 and 57. Power source 57 supplies current to the second rail 69. Power source 67 supplies current to the third rail 65.

The data from the first and second thermistor correlate and estimate the voltage drift of the sensors. Preferably, the thermistors are of NTC Type and may require a calibration step to be include in the start-up procedures of the connected controller.

The present embodiment, uses a current source to actively anticipate the temperature of the air path rather than the prior art examples which rely on the voltage drop across a single thermistor.

The first rail 64 is preferably connected to a first P-CH Switch 60. This P-CH Switch 60 may be a MOSFET transistor switch wherein the P channel is used as the switching path. The switch 60 is connected to an over temperature circuit 61 which in turn connected to the output of buffer 55.

Preferably, the fourth rail 66 may be connected to a first N-CH Switch 59. This N-CH Switch 59 may be a MOSFET transistor switch wherein the N channel is used as the switching path. The switch 59 is connected to a ground connection marked GND.

Preferably, the third rail 65 is connected to current source 68 and a first buffer module 55. The buffer module 55 is then connected to first and second shifter circuits 54 and 53. The buffer module 55 is also connected the output of the over temperature circuit 61. It is noted that the buffer modules may function as amplifier circuits or modules.

Between the power source 67 and the second rail 69, a second buffer module is connected and then in turn connected to a third shifter circuit 52.

The outputs of the first, second and third shifter circuits 54, 53, and 52 allows for the master control unit 51 to determine air path temperatures and control the heating elements 61 in the conduit.

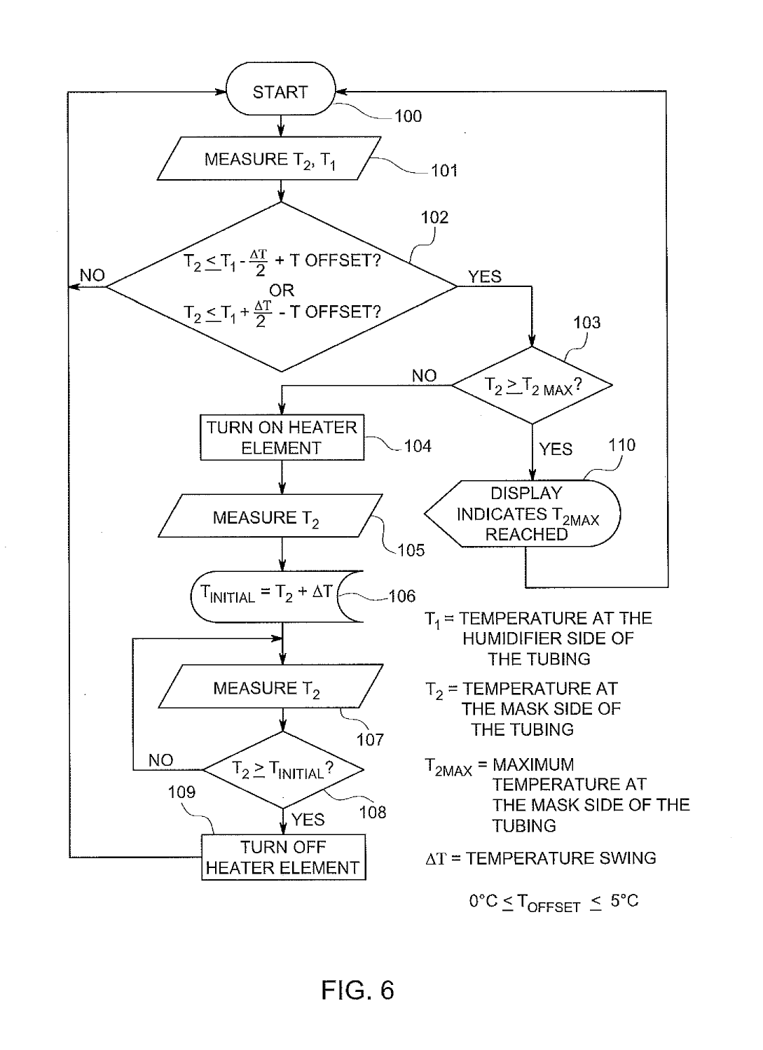

FIG. 6 depicts a process to be used as part of the system to control and monitor the temperatures within the air path of the respiratory conduit. Preferably, this flowchart may be implemented as a software program or firmware installation operating on the controller or master control unit 51.

The process begins at step 100 labelled ‘start. Step 101 receives input data to measure the measured temperatures experienced by the first and second thermistor 62 and 63. These measured and calculated temperatures are respective recorded in the system as T₁ and T₂. It is noted that T₁ is measured from second thermistor 63 and T₂ is measured from first thermistor. Preferably, the predetermined starting condition for the heating elements is turned off.

In step 102, T₁ and T₂ are independently compared to each other using the calculation shown in FIG. 6. T_{offset} is a predetermined number between 0 and 5 degrees Celsius that is recorded in the controller prior to operation of the unit. T_{offset} is preferably set by a user and can be amended. It is a measure of the reasonably acceptable temperature differential between the first and second thermistors. If the temperature close to patient interface is lower than the temperature proximal to PAP device by significant degree (as determined by the aforementioned algorithm) then the process proceeds to step 103 otherwise the process loops back to step 100.

Step 103 determines whether the temperature proximal to the patient interface has already exceeded a predetermined safety limit temperature of T₂ₘₐₓ. If the safety temperature is exceeded, the P-CH Switch 60 will be turned off and the process displays a warning message (as per step 110) and loops back to step 100; otherwise the process or system proceeds to step 104.

Step 104 is wherein the controller turns the heating element on. In step 105, the controller remeasures T₂. The controller then calculates a variable Tᵢₙᵢₜᵢₐₗ based on the immediate T₂ plus the Temperature Swing Variable.

The controller remeasures T₂ in step 107 and then in step 108 compares the newly measured T₂ to T initial. If the temperature at second thermistor has not increased by the threshold of T initial which is equal the first measured value of T₂ plus the temperature swing then the system continues to loopback to step 107 and increase temperature. When the condition of step 108 is satisfied the controller proceeds to step 109, wherein the heating element is deactivated.

The system loops back to the step 100 upon completion of step 109.

In a further embodiment of the present invention, the overall system or device includes: a respiratory conduit 41, a PAP device and a patient interface. Preferably, the system may also include a humidifier (not shown) but the preferred humidifier is mounted on or within the patient interface.

More preferably, the humidifier is adapted to form a humidity and moisture exchanger (HME) module which is mounted within the patient interface or mask. The HME module may be a replaceable and disposable cassette module which may be mounted or positioned within the body or cavity of the mask. The HME module preferably acts to filter microbes and dust/debris particles from the airflow; and also capture and recycle moisture. It is noted that the patient ̆s airflows usually contain a humidity of about 90% and rapid cooling of the air exiting the patient ̆s respiratory path may result in rainout occurring in the patient interface. The HME module may preferably function to absorb and reuse the moisture exiting the patient. Preferably, the HME module should be received heated air from the conduit.

The preferred system using the HME module delivered dry heated air to the patient interface as the associated PAP device in this embodiment is not required to include a separate humidification system or device. The dry heated air in the conduit is re-humidified as it passes through the HME module.

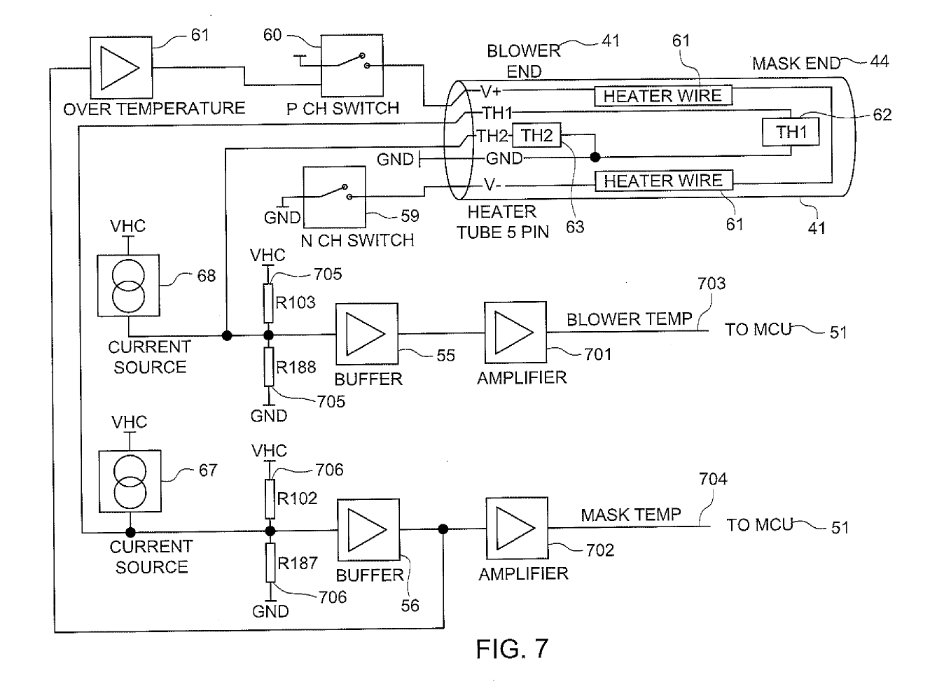

The heating system of this second preferred embodiment is depicted in FIGS. 7 to 11. Specifically, in FIG. 7 a heating system has been provided that is modelled from the previous embodiment shown in FIG. 5 and uses similar numbering and features.

However, FIG. 7 differs in a key areas or regions of understanding. The conduit 41 has been modified to use a 5 pin or five electrical connector configuration. Four electrical wires are encapsulated within the conduit 41 for the overall heating system. Preferably, the four encapsulated wires are wound in a helical pattern around the conduit and encapsulated within its design.

The heating system includes a first thermistor 62 mounted or positioned near to a first end 44 of the conduit 41. The first end is preferably adapted or modified to engage or secure to a patient interface (not shown). The patient interface may preferably contain a HME module.

A second thermistor is encapsulated within the conduit 41 at or near to the second end 41 of the conduit 41. The second end is adapted to engage or secure against the air outlet of a blower device, as called the PAP device (not shown). Preferably, the blower does not include a humidifier.

Preferably, the first and second thermistors 62, 63 are electrically isolated from the heater 61 within the conduit 41. The first and second thermistors 62, 63 are electrically connected on a first circuit and the heater 61 is electrically connected on a second circuit. The first and second circuits are not connected within the conduit which may improve the accuracy of the sensing circuit which is formed by the first circuit.

The second circuit may be exclusively used for heating the heater 61 and the heater 61 may be driven by variable DC voltage supply applied to the second circuit.

In FIG. 7, the acronym MCU stands for master control unit which is the preferred processor to be mounted within the housing of the PAP device or blower device. Preferably, the system allows for the accurate monitoring of temperatures at both ends of the conduit with reduced relative error.

FIG. 7 allows for the heating system to output mask temperature signals 704 and blower temperature signals 703 to the MCU 51. The MCU 51 may use the data acquired from these signals to automatically adjust the variable DC voltage supply applied to the heater 61 to improve the delivery of heated air to the patient interface.

Preferably, the system may additionally include resistor sets 705, 706 to adjust the rail voltage or current. In one embodiment, the output voltage operates within the range of 0 to 3V for operational temperature of 10° C. to 40° C. Preferably, in another embodiment, the output voltage operates within the range of 1 to 2.4V for an operational temperature of 0° C. to 50° C.,

Amplifiers 701 and 702 may be included upstream of the blower and mask temperature signals 703 and 704 to improve the signal quality and amplitude.

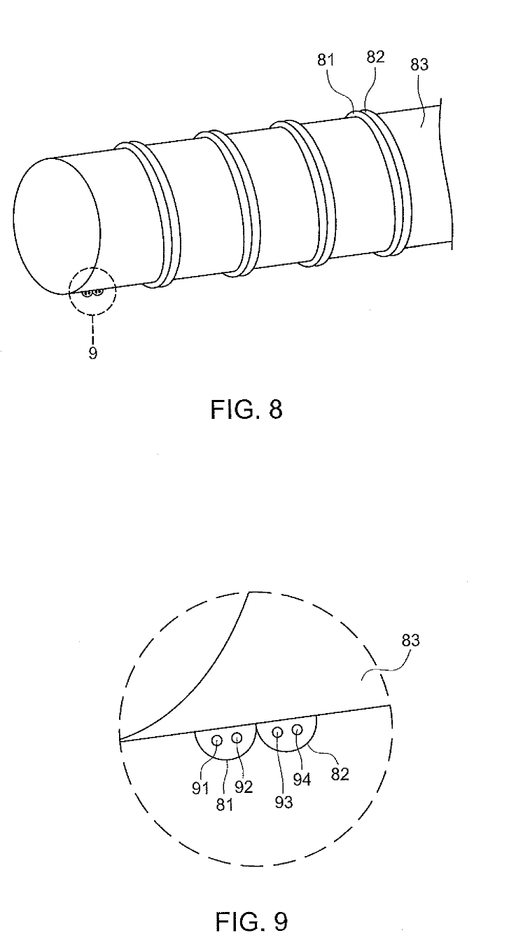

The heating system within this embodiments preferably designed to check the operation of the Tube Heater controller circuitry as further described in FIGS. 8 to 11. This circuitry may be designed to be incorporated within the Main PAP controller or blower PCB (printed circuit board). The Tube Heater Circuit may operate by continuously measuring the temperature at both ends of the tube and by powering the heater wires accordingly.

The P channel Mosfet is typically always on but will turn off in an over-temperature condition where the mask end thermistor measures a temperature higher than the preset maximum temperature limit The limit may be 40° C. or more.

The N channel switch is used to turn the heater tube on and off to regulate the temperature to the set level.

Additionally, a section of a preferred respiratory conduit 83 is shown or depicted in FIGS. 8 and 9. In FIG. 8, the respiratory conduit 83 includes two sets of helical wound wires 81 and 82. In this embodiment, two sets of wires are shown but other combinations of wiring sets are possible and may be within the scope of this present invention.

The preferred conduit 83 as shown in FIG. 8 is depicted without the patent interface or the CPAP flow generator and additionally the mounting clips and securing means have been removed for the illustration.

In this embodiment, the preferred respiratory conduits includes at least two wiring sets and at least 4 wires wherein at least some of the wiring sets and wires are encapsulated within the outer surface of the conduit.

The helical configuration of the winding of the wires allows for greater or improved flexing of the conduit without the said flexing causing undue damage to the wires forming the wires sets.

In this embodiment, the two wiring sets are encapsulated within an over-moulding of a polymer which may include silicone polymer. Preferably the two wiring sets are spaced apart from each other and wound in parallel fashion around the circumference of the conduit to form a double helix pattern.

The two wiring sets are spaced apart by a relatively small gap when compared to the next level of winding forming the helix. The small gap may be within the vicinity of 1-2 mm.

Each wiring set preferably includes two wires each. The first wiring set 81 may include first wire 91 which adapted to carry −ve signal for a heater wire incorporated into the conduit, the second wire 92 is adapted to carry sensor information and signals. Alternately, wire 92 may be used to carry the sensor GND.

The second wiring set includes third wire 93 and fourth wire 94. The third wire 93 is adapted to carry +ve signal for the heater wire incorporated into the conduit. The fourth wire 94 is adapted to carry signal or information to and from a remote temperature sensor located or proximal to the earlier described patient interface. Preferably the first and third wires are adapted to be joined or electrically connected at the end of the conduit proximal or near to the patient interface or mask.

Further, it may be advantageous to separate the four wires into two separate wiring sets to improve flexibility of the configuration and also to ameliorate or lessen the likelihood of wire failure or short circuit in the event that the encapsulation over the wiring sets is breached.

Preferably, the wires in the wiring sets may be constructed of conductive metal including copper wiring. The conduit may be generally constructed of a flexible polymer including silicone polymer or similar flexible polymeric material.

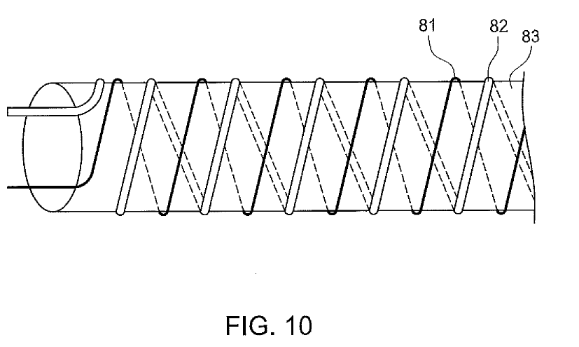

A further preferred embodiment is illustrated in FIG. 10, wherein a cross sectional view of a respiratory conduit 83 is shown. The conduit 83 is a hollow tube adapted to carry pressured air between a flow generator or blower (not shown) and patient interface (not shown).

The outer surface includes two sets of wires, similar to FIGS. 8 and 9, wherein the first set 81 and second set 82 of wires have been again encapsulated within the outer surface of the conduit 83. However in this embodiment, the first set 81 and second set 82 have been offset from each other and are preferably 180 degrees out of phase with each other. The overall configuration depicts the first and second sets of wires forming substantially a double helix pattern around the outer surface of the conduit.

FIG. 10 may provide the additional advantage of more uniform heating of the conduit 83 as both the first and second wiring sets include heated wires as similar wires have been used as per FIGS. 8 and 9.

Further, one of the benefits of separating the wiring sets encapsulated within the conduit is to prevent or lessen the risk of short circuit or arcing between the heater wires from leading to a condition wherein extra current is drawn from the 24V supply to ground, and thereby further causing the heated conduit to overheat. Overheating may lead to damage of the overall medical device or personal injury to the patient using the device and should be avoided or limited, if possible.

Please note for purposes of this disclosure, Voltage H eater Controller (VHC) may mean the analogy power supply to the heated tube controller thermistor. Generally, VHC may operate at about 3.3 Volts.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. A system for heating a respiratory conduit positioned between a PAP device and a patent interface, wherein the system comprises:
a first thermistor encapsulated within the conduit proximal to a first end of the conduit which is proximal to the patient interface;
a second thermistor encapsulated within the conduit proximal to a second end of the conduit which is proximal to the PAP device;
a heater encapsulated within the conduit; and
wherein the first and second thermistor are electrically isolated from the heater within the conduit.

2. (canceled)

3. The system of claim 1, wherein the conduit includes a mating portion for connecting with the PAP device or a humidifier device, the mating portion including electrical contacts for connecting the wires encapsulated within the conduit with another set of corresponding electrical contacts on the PAP device or humidifier device.

4. The system of claim 1, wherein the first thermistor is mounted in a molding extending inwardly within the conduit for measuring temperature proximal to the patient interface.

5. The system of claim 1, wherein the second thermistor is mounted in a molding extending inwardly within the conduit for measuring temperature proximal to the PAP device.

6. The system of claim 1, wherein the first and second thermistors are electrically connected to a first circuit; and the heater is electrically connected to a second circuit and wherein the first and second circuits are not electrically connected within the conduit.

7. The system of claim 1, wherein the system comprises a humidifier mounted with or on the patient interface.

8. (canceled)

9. (canceled)

10. A control system for a heated respiratory conduit positioned between a PAP device and a patient interface, wherein the control system comprises:
a power supply to provide power to at least one heating element encapsulated in a conduit;
an over temperature control circuit to prevent overheating of the heating element;
a heating control circuit configured to control the heating element; and
a sensing circuit including at least a first thermistor positioned in the respiratory conduit proximal to a patient interface and configured to indicate a second temperature, and a second thermistor positioned in the heated conduit proximal to a PAP device and configured to indicate a first temperature.

11-14. (canceled)

15. The system of claim 10, wherein the system compares the second temperature to the first temperature and when the second temperature is lower than the first temperature by a function of a predetermined offset temperature the heating control circuit is activated.

16. The system of claim 15, wherein the predetermined offset temperature is adjustable between 0 and 5 degrees Celsius.

17-19. (canceled)

20. A process for controlling the temperature of air inside a respiratory conduit positioned between a PAP device and a patient interface, wherein the process comprises the following steps:
a first temperature is measured by a second thermistor positioned proximal to the PAP device and a second temperature is detected by a first thermister positioned proximal to the patient interface; and
comparing the second temperature to the first temperature and when the second temperature is lower than the first temperature by a function of a predetermined offset temperature at least one heating element within the conduit is activated.

21. The process of claim 20, wherein the predetermined offset temperature is between 0 and 5 degrees Celsius.

22. The process of claim 21, wherein the heating element continuously heats the conduit until the value of the second temperature exceeds the sum of the initial second temperature measurement and a temperature swing value.

23. (canceled)

24. The system of claim 1, wherein the conduit comprises wires encapsulated within an outer surface of the conduit for electrical connection with the first thermistor and/or the second thermistor.

25. The system of claim 1, wherein the heater includes a wire encapsulated within an outer surface of the conduit that generate heat when an electrical current is applied.

26. The system of claim 1, wherein the conduit comprises wires encapsulated within an outer surface of the conduit in a helix.

27. The system of claim 1, wherein the conduit comprises at least two spaced apart wire sets encapsulated within an outer surface of the conduit in a helix.

28. The system of claim 1, wherein the heater is powered by a variable DC voltage power supply.

29. The system of claim 10, wherein the over temperature control circuit deactivates the heating control circuit if the second temperature exceeds a predetermined maximum temperature.

30. The system of claim 10, wherein the power supply is a variable DC voltage power supply.

31. The system of claim 10, wherein the system deactivates the heating control circuit when the value of the second temperature exceeds the sum of the initial second temperature measurement and a temperature swing value.
